(19) United States
(12) Patent Application Publication
(10) Pub. No.: US 2021/0295962 A1
(43) Pub. Date: Sep. 23, 2021

(54) Electronic Patient Registration and Billing

(71) Applicant: Melissa Bloom, Clinton, MS (US)

(72) Inventor: Melissa Bloom, Clinton, MS (US)

(21) Appl. No.: 16/821,756

(22) Filed: Mar. 17, 2020

(51) Int. Cl. G16H 10/60; G16H 40/20; G06Q 50/22; G06Q 40/08; G06Q 10/10; G06Q 10/02; G06K 9/00

(52) U.S. Cl. CPC G16H 10/60; G16H 40/20; G06Q 50/22; G06K 9/00288; G06Q 10/1095; G06Q 10/02; G06Q 40/08

(57) ABSTRACT

The present invention relates to new and useful improvements in data collection, synchronization, storage and handling of a patient registration, direct, track, and optimize the efficiency of patient activity and patients to have ready access to their status to control and healthcare providers have surety of the process being complete, accurate and ready for revenue cycle process.

### BACKGROUND

### Copyright Notice

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to the fields of healthcare and information technology. More particularly, the invention relates to automating a system for registering patients with a complete demographic and insurance profile, check in and check out process in a healthcare facility.

### DESCRIPTION OF THE RELATED ART

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Healthcare providers, such as physicians, create large volumes of patient information during their business at healthcare facilities, such as hospitals, clinics, laboratories and medical offices. For example, when a patient visits a physician for the first time, the physician generally creates a patient file including the patient's demographic, current treatments, medications, insurance and other pertinent information. This file generally includes the results of patient visits, including laboratory test results, the physician's diagnosis, medications prescribed and treatments administered. During the course of the patient relationship, the physician supplements the file to update the patient's medical history. When the physician refers a patient for treatment, tests or consultation, the referred physician, hospital, clinic or laboratory typically creates and updates similar files for the patient. These files may also include the patient's billing, payment and scheduling records.

Healthcare providers can use electronic data processing to automate the creation, use and maintenance of their patient records. For example, Schneiderman discloses a computer system for recording electrocardiograph and/or chest x-ray test results for a database of patients in U.S. Pat. No. 5,099,424. In U.S. Pat. No. 4,315,309, Coli discloses a patient report generating system for receiving, storing and reporting medical test data for a patient population. Mitchell, in U.S. Pat. No. 3,872,448, likewise discloses a system for automatically handling and processing hospital data, such as patient information and pathological test information using a central processing apparatus. In U.S. Pat. No. 5,065,315, Garcia discloses a computerized scheduling and reporting system for managing information pertinent to a patient's stay in the hospital. However, these electronic data processing systems cannot handle patient data in the wide variety of data formats typically produced by healthcare providers, such as physicians, laboratories, clinics and hospitals. Wilkins U.S. Pat. No. 6,523,099 relates to an individualized patient medical record system, where the patient can later review his or her own medical records. The patient carries a digital record on his or her person, with the entire medical record being stored on a portable data memory device, a CD-ROM.

Existing systems for automating healthcare don't focus on patients lack of understanding the registration process and they often have to endure long waiting times and poor customer service and consistent follow-up communication in order to have a compete, billable profile. Likewise, there is not a focus on the knowledge level a front desk worker has to have to guide the patient into completing a complete patient registration record. Prior art shows the gathering and storage of identification and insurance cards sent to staff on the back end for analysis and entry into the billing system. To alleviate this problem, a system is needed that both maximizes the efficiency and accuracy of the healthcare process and provides patients easy access to their status and/or control of the process. This system also alleviates the problem of having unqualified personnel completing this vital process by using artificial intelligence to guide the data collection process and map it properly. None of the prior art methods have been found to be completely suitable to meet these needs and are cumbersome. The present invention provides such a method and the overall combination of these features is nowhere disclosed in the prior art cited above which appears to be representative of the general art in this area although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, and abstract as a whole.

So as to take care of the above cited issues, the technique and arrangement of this innovation centers to a product/software that will sort out and mechanize the patient registration process in a way that requires no mediation from the staff of the clinical office. Furthermore, it will empower correspondence legitimately between the patient and the clinical office's practice management and/or EMR, and eliminate the chance of manually transferring wrong data, missing pertinent data and time and labor cost.

The system will go after explicit billing data that guarantee appropriate submission of clinical cases for installment to the appropriate healthcare provider. Subsequently making a profile that is finished, exact and prepared to get billable charges. The product will use the camera or comparable gadget to see, scan, read, concentrate and extract explicit information from the patient's officially sanctioned ID and insurance cards. The programming language is installed with the information on both a broadly knowledgeable front desk worker and clinical medical biller with the ability to send directions and solicitations for data to the patient by means of the tablet dependent on the patient's choices and the filtered information.

Another embodiment of the present invention is that the electronic registration record arrangement of the present innovation mechanizes and streamlines existing techniques for patient profile creation, maintenance and retrieval. Rather than different frameworks, the present development makes and keeps up every single patient datum electronically and along these lines can take out or supplement making and keeping up of physical information records. The framework empowers to catch and break down patient information rapidly and effectively. Utilizing the present creation, medicinal services suppliers enter tolerant information quickly at the point of care.

Another object is to provide a novel system of the present invention which includes the capability to manage a wide variety of patient data formats and may include patient data from external sources, such as laboratories, pharmacies, outside healthcare providers and insurance cards.

It is another object of this invention to provide authorized healthcare providers can access a record while other providers use the same record allowing for real-time collaboration without manipulating the information. The EMR system enables enhanced analysis of patient data by providing access to reference databases for diagnosis, procedures and medication.

Briefly, it is a principal object of the invention to make sure patients are properly enrolled and registered with a complete demographic and insurance information with ability to perform this task via kiosk or mobile device requiring no medical staff intervention and extensive knowledge to complete the registration task. Besides, the framework empowers upgraded investigation of patient information by giving access to reference databases to determination, systems and prescription.

It is the principal objects of the invention to make sure patients are properly enrolled and registered with a complete demographic and insurance information with ability to perform this task via kiosk or mobile device requiring no medical staff intervention and extensive knowledge to complete the registration task. Additionally allowing for completion of check out tasks inclusive of but not limited to payment of copay and rescheduling of follow-up visits. Besides, the framework empowers upgraded investigation of patient information by giving access to reference databases to determination and systems

Features of the invention are that the system improves patient satisfaction by decreasing hold up times to be seen by a clinician and improving communication to the patient and hospital management and drastically improves the data collection process for both the front desk and billing personnel helping to further secure revenue flow.

Still another part of the present development incorporates a strategy for communicating with an outside source having an interface to an electronic practice management or electronic records framework, including the means of finding an interface for the outer source, associating with the outer source utilizing the interface, and changing over patient information for movement between the outside source and the electronic clinical records framework.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 100 is a flowchart illustrating the process flow of the system of the present invention showing a complete and accurate patient demographic, insurance and billing profile ready to receive billable charges for a new patient.

FIG. 200 is a flowchart illustrating the process flow of the system of the present invention showing a complete and accurate patient demographic, insurance and billing profile ready to receive billable charges for an existing patient.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

Unless otherwise defined, all terms (including technical and scientific terms) used herein have the same meaning as commonly understood by one having an ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques are disclosed. Each of these has individual benefit and each can also be used in conjunction with one or more, or in some cases all, of the other disclosed techniques. Accordingly, for the sake of clarity, this description will refrain from repeating possible combination in an unnecessary fashion. Nevertheless, the specifications and claim/s should be read with the understanding that such combinations are entirely within the scope of the invention and the claim/s.

The method of the present invention is that it relates to a software that can be introduced on a Windows operating or macOS. It will likewise have a mobile application component. This will permit the patient to finish the patient enrollment process face to face at a booth enveloping a tablet or across the board PC with a camera or on their cell phone if they want to finish the enlistment procedure before their arrangement date.

In some embodiments, the invention relates to a number of options to encourage check-in to the facility by the use of an identification device (e.g., a bar-coded loyalty or identification card). Check-in can be facilitated with the identification device or any other suitable identifying method, e.g., biometric (e.g., fingerprinting) or electronic strategies.

The activity of the framework can be outlined through a common patient visit to a social healthcare facility, for example, an emergency clinic or center. The framework starts catching information on tolerant stream when the patient shows up at the healthcare facility and directs persistent development from enlistment through treatment to takeoff. The framework can play out its underlying catch direct from a patient's PC before the patient's landing in the healthcare facility.

The software will show the patient the medical practice's financial policy, consent to treat policy and medical record release policy for review that would be updated from time to time consultations or through the data taken at the time of enrolling.

The system includes methods of communicating patient information to external entities. For example, e-mail notifications can be sent to external entities and business partners indicating that a patient has been registered at the facility.

The way that this organized information is changed would naturally synchronize to all the clinical connected data, patient selection and patient status real time within the EHR/EMR or PMS. Backend onboarding support will further assist healthcare facilities in ensuring proper setup and customized needs.

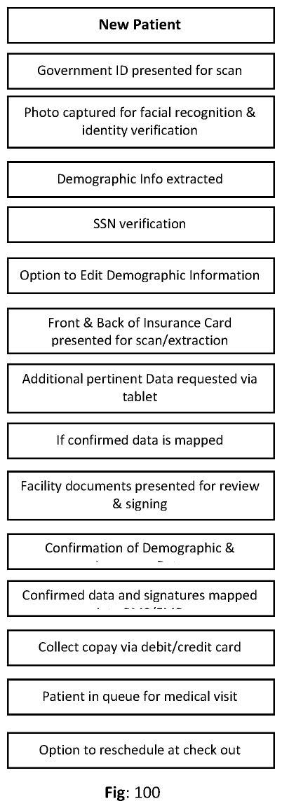

FIG. 100 shows a mechanism to setup data and to streamline information to be used in future. The product demands persistent enrollment by introducing government provided ID card to camera to be checked for photograph and to extricate explicit demographic information. The software will ask the patient to position in front of the camera for a photo to be used on profile and labeled as on “identification” card.

The system will actuate facial acknowledgment programming to make a correlation for coordinating the live photograph and government provided distinguishing proof card to wipe out possibility of identity fraud. The identification card is examined and read by means of the camera. The patient's name, date of birth, sex and address are extricated from the card to be mapped straightforwardly into the comparing fields into the corresponding fields into the existing practice management system. The data entered is verifiable and can be edited if there is an anomaly. The system also uses insurance details of the patient to be stored and later retrieved upon need. Once all the information is verified to be correct the data will be mapped directly into the corresponding fields in the database system. In-future the software scans and extricates information from the insurance card automatically using front and back of the insurance card so to build the patient registration profile. The software will also have medical facility's consent to treat, financial agreement and release of records policy for the patient to review and agree. The software will request a signature and then map that signature to the corresponding area for each of the aforementioned forms. The patient will have also option to pay for the service using saved credit or debit card details.

Once the payment is made the patient has the option to check in immediately or check in later. If check in later then the system will automatically allocate time and day. If check in now the system will use facial recognition software to enter the information of the patient and a notification will be sent to nurse or assigned person that requires acknowledgment so that the patient is put in queue to be seen by the needed doctor. The system will also make the process for rescheduling of appointment simpler and confirmations can be sent using the automated system for the upcoming appointment.

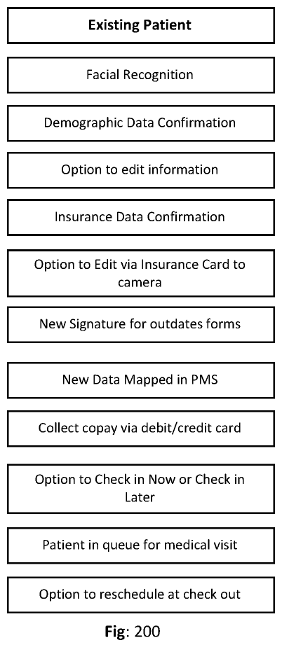

FIG. 200 shows process for on-going patient where a patient is first of recognized using facial recognition system and all the information is retrieved. If there is anything that need to be updated the system gives the patient ability to modify the detail before finalizing it. The old data is replaced with the new data and is mapped in those fields. Then the remaining process is same where the user has option to check in immediately or check in later and the same process follows as discussed in FIG. 100.

Another embodiment of the invention is that the system educes major administrative duties in healthcare thus streamlining the process. The system is made to interface with existing EHR/EMR and PMS live already like, but not limited to Practice Fusion and NueMD.

In addition the system allows for 100% complete registration on a mobile device or at point of service at designated kiosk housing a camera enabled PC or tablet.

The initial screen with the software is activated presents 3 options: NEW PATIENT, EXISTING PATIENT, CHECK IN and CHECK OUT. The check-out option is disabled on the link that allows the patient to register via mobile device.

For NEW PATIENT the process is explained as following:
a) If new patient, software requests patient present government issued identification card to camera to be scanned for photo and specific demographic data.
b) The software will then ask the patient to position in front of the camera for a photo.
c) The system will activate facial recognition software to make a comparison for matching the live photo and government issued identification card to eliminate chance of identity theft. The live photo is stored as part of the patient's profile for subsequent check-ins. The identification card is stored in the patient documents area of the patient's profile of the practice management system and labeled as “identification” card.
d) The identification card is scanned and read via the camera. The patient's name, date of birth, gender and address are extracted from the card to be mapped directly into the corresponding fields into the existing practice management system.
e) The software then activates the keypad on the tablet and requests the patient's social security number and telephone number be entered. Email address is optional based on facility preference.
f) A summation of the data scanned and entered shows on the screen to allow patient to confirm as accurate or edit.
g) The patient has the option to touch the address area and edit if the address pulled from the driver's license is no longer current.
h) Once the patient confirms via the screen that all the data is accurate and selects done, all of the data is mapped into the corresponding fields directly into the interfaced practice management system.
i) The software asks the patient via the pad if he or she has insurance to select yes or no.
j) If no, the software will set the patient to a self-pay profile
k) If yes, the software will request the patient to present the insurance card to be scanned front and back.
l) The software scans, reads and locates the payer ID first to activate existing insurance profile in the practice management system. This also ensures the correct payer is already mapped and will be sent to the correct payer.
m) The software then scans and reads for the policy number, group number and applicable copay, then maps the information into the corresponding fields directly into the practice management system.
n) The insurance company address and customer number is located and read by the software to ensure that what's on the existing insurance profile matches and does not require update.
o) If the address and phone number are not a match, they will be noted to the patient's account for manual clarification.
p) If the subscriber name and/or policy holder on the insurance card does not match the name of the patient, the patient will be asked to select from a drop down menu their relationship to the policy owner by the software via the screen keypad.
q) The software will then ask the patient to enter the policy holder's date or birth and confirm whether or not their address is the same or different. If it is different, the software will activate and show the tablet to allow the patient to enter it.
r) If the patient's coverage is worker's compensation, the details on the policy number, the date of injury and the name and address of their employer will be requested via the keypad and mapped directly into the worker's compensation case profile.
s) A summation of all of the data scanned on the insurance card and entered by the patient will appear on the screen for the patient to confirm all correct.
t) Once done is selected by patient to confirm accurate, the data will be mapped directly into the corresponding fields directly in the interfaced practice management system.
u) The insurance card image, front and back, will be stored into the patient documents area of the patient's profile in the practice management system and labeled as “insurancecardfront” and “insurancecardback” with the scanned date.
v) If the patient is under the age of 18, the software will request their government issued identification card of the guarantor be presented for scan and read. The guarantor's name, address, date of birth will be extracted and mapped into the corresponding fields into the practice management system. The software will ask the patient or guarantor to enter their social security number and phone number via the keypad on the screen. The patient will be asked to position in front of the camera for their profile picture. If the guarantor is present, the software will also prompt the guarantor to position in front of the camera for a photo. All of entered and or scanned and extracted data will be presented in summation on the screen to be confirmed as accurate. The guarantor and/or patient will have the option to modify the address directly on the keypad if the address extracted from the identification card is not current, once confirmed, all of the data will be mapped into the corresponding fields of the guarantor profile area for the patient in the practice management system. The guarantors identification card image will be stored in the patient documents area of the practice management system and labelled as “guarantoridentificationcard”. If the patient does not have a government issued ID card, the software will prompt the patient to enter all of their demographic information in field by field, including but not limited to, first and last name, date of birth, gender, address, phone number and email address
w) Based on the specific facility's set-up, it is optional here to the patient to enter a person as an emergency contact. The screen will populate allowing the patient to enter selected emergency contact's name, address, phone number and relationship to them. There is an area in over 90% of existing practice management system's software to house this information. Therefore the same as above data sets, the information will be mapped directly into the practice management software in the corresponding fields.
x) The software will again present the demographic, insurance and guarantor, if applicable individually on the screen with an arrow to move through each for the patient to review and confirm as accurate.
y) The software will then present the medical facility's consent to treat, financial agreement and release of records policy for the patient to review and agree. If the facility has other specific policies, they can be loaded as well. The software will request a signature and then map that signature to the corresponding area for each of the aforementioned forms.
z) If an applicable copay was identified, read and mapped from the insurance card, the systems will ask the patient for payments via credit/debit card at that time.
aa) The patient will have the option to store the card for future use—optional
bb) Once the payment is secured, the patient has the option to: CHECK IN IMMEDIATELY or CHECK IN LATER.
cc) The check in later option is for patients that elect to register prior to coming into the office via their mobile device.
dd) Those patients will have the option to select CHECK IN from the welcome screen at the time they actually present for their appointment.
ee) Once that option is selected, the camera will activate, capture their facial image using facial recognition software, locate their profile and automatically check them in as they will have already completed and provided the pertinent details and payment via their mobile device.
ff) Once the patient is checked in, whether registered by way of onsite registration or prior mobile registration, a notification will be sent to nurse or assigned person that requires acknowledgement so that the patient is put in queue to be seen.

For EXISTING PATIENT the process is explained as following:
a) If the patient selects existing patient, the camera activates and asks the patient to position in front of it.
b) The software deploys the facial recognition component, and uses image match and recognition to activate presumed accurate patient's profile.
c) The name on the matching profile presents and the patient is asked to enter the last 4 digits of their social security number to confirm, unlock and fully activate the profile as a 2-factor level of security.
d) The software presents a summation of the patient's current demographic data on the screen making the address and phone number editable if necessary. Email address is optional based on facility preference.
e) The patient can confirm via the screen that the information is accurate. If no is touched, the patient can edit the address and phone number directly on the screen via the keypad.
f) Once the patient selects done that confirms all demographic data is accurate, the old data is stored and new data is mapped and replaces the data in those fields in the interfaced practice management system.
g) The software then presents a summation of the patient's current insurance data for the patient to confirm as accurate.
h) If accurate, the system will check the date of the last signature captured in the office documents. If it has been a day or more over a year or a new form has been uploaded, they will be presented on the screen for review for agreement and a signature will be requested via the screen. The signature and date will be mapped to the appropriate area of all forms.
i) If the patient does not confirm as accurate, the software will ask the patient if he or she has insurance. If no, the software will set the patient profile to self-pay and ask the patient to confirm.
j) If the patient indicates via the screen that he or she does have insurance; the software will request the patient present the insurance card, front and back to the camera. The software then scans, reads and locates the payer ID first to activate existing insurance profile in the practice management system. This ensures the claims are already mapped and will be sent to the correct payer.
k) The software then scans and reads for the policy number, group number and applicable copay, then maps the information into the corresponding fields directly into the practice management system.
l) The insurance company address and customer number is located and read by the software to ensure that what's on the existing insurance profile matches and does not require update.
m) If the address and phone number are not a match, they will be noted to the patient's account.
n) If the subscriber name and/or policy holder on the insurance card does not match the name of the patient, the patient will be asked to select from a drop down menu their relationship to the policy owner by the software via the screen keypad.
o) The software will then ask the patient to enter the policy holder's date or birth and confirm whether or not their address is the same or different. If it is different, the software will activate and show the tablet to allow the patient to enter it.
p) If the patient's coverage is worker's compensation, the details on the policy number, the date of injury and the name and address of their employer will be requested via the keypad and mapped directly into the worker's compensation case profile.
q) A summation of all of the data scanned on the insurance card and entered by the patient will appear on the screen for the patient to confirm all correct.
r) Once done is selected by patient to confirm accurate, the data will be mapped directly into the corresponding fields directly in the practice management system. The new card will be saved as “insurancecard.currentdate1 and insurancecard.currentdate2—front and back.
s) The software will again present the demographic, insurance and guarantor, if applicable individually on the screen with an arrow to move through each for the patient to review and confirm as accurate. The system will also check the date of the last signature of the office forms and the date stamp of the last signature is 366 plus days old, the forms will be represented to the patient for review and signature. If new signature is required, it will replaced the old ones and be mapped in the PMS with the current date.
t) If a copay was identified, read and mapped from the insurance card, or there is a balance due from prior care, the system will ask the patient for payments via credit/debit card at that time. If copay was identified, read and mapped from the insurance card, the system will ask the patient for payments via credit/debit card at that time.
u) The patient will have the option to store the card for future use—optional
v) Once the payment is secured, the patient has the option to: CHECK IN IMMEDIATELY or CHECK IN LATER.
w) The check in later option is for patients that elect to register prior to coming into the office.
x) Those patients will have the option to select CHECK In from the welcome screen at the time of their appointment.
y) Once that option is selected, the camera will activate, capture their facial image using facial recognition software, locate their profile and automatically check them in as they will have already completed and provided the pertinent details and payment via their mobile device.
z) Once the patient is checked in, whether registered by way of onsite registration or prior mobile registration, a notification will be sent to nurse or assigned person that requires acknowledgement so that the patient is put in queue to be seen.

For CHECK OUT the process is explained as following:
a) The system allows for the option for the patient to CHECK OUT once the appointment is complete.
b) The patient will select the CHECK OUT option and reschedule option will appear.
c) Depending on the medical facility's set up and resources, the options will appear to which the patient can reschedule. Those options will be, but are not limited to, rescheduling with the doctor seen that day, another doctor within that practice of the lab. The available days and times will populate for the patient to select based on preset options.
d) Once the patient makes the selection for time and date, the software will send an appointment confirmation with option to add to calendar to their mobile device or email based on facility's patient profile data sets.
e) If the patient is referred out and the appointment is scheduled by the medical facility, the patient may be asked to sit before check out to allow that to be completed. Once its put in the system, when the patient goes to check out, the notification of the appointment will appear on the screen and patient is advised that facility name, address, phone number, appointment time and date will be sent to their mobile device or email.
f) Once complete, a customizable thank you message will appear for the patient and the screen will reset to the initial screen for the next patient.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1) A software system that read and extracts data from both the identification card and the front and back of the insurance card; communicates and maps directly into the existing practice management and/or EMR system requiring no human entry as the normal now comprising:
a. device camera and keypad for input or modification of data;
b. patient data and picture capture to enter information provided by a patient,
c. automated and accurate patient demographic and complete insurance registration, ready for billable charges:
d. accurate and complete registration via a kiosk or a computer within the medical facility or via mobile device owned by the patient.

2) The system allows check-in with payment option, complete registration and checkout with option to schedule next appointment at a kiosk housing a computer/tablet device with camera OR a mobile hand held device like cell phone.

3) The software that will allow accurate and complete registration at a point of care system to capture patient data using facial recognition mechanism and to schedule and re-schedule appointments that can be communicated through a mobile application.

4) The software will allow for extensive customization in flow based on individual practice preferences, onboarding assistance inclusive but not limited to proper creation and set up of insurance profiles.
